(19) United States
(12) Patent Application Publication
(10) Pub. No.: US 2010/0036256 A1
(43) Pub. Date: Feb. 11, 2010

(54) OFFSET ULTRASONIC HAND PIECE

(76) Inventors: Mikhail Boukhny, Laguna Niguel, CA (US); James Chon, Irvine, CA (US)

Correspondence Address: ALCON, IP LEGAL, TB4-8, 6201 SOUTH FREEWAY, FORT WORTH, TX, 76134, US

(21) Appl. No.: 12/188,689

(22) Filed: Aug. 8, 2008

(51) Int. Cl. A61B 8/00

(52) U.S. Cl. 600/459

(57) ABSTRACT

An ultrasonic hand piece has a horn, piezoelectric crystals, and a cutting tip. The piezoelectric crystals and cutting tip are coupled to the horn. A centerline of the piezoelectric crystals is offset from a center line of the cutting tip such that oscillatory movement is produced in the cutting tip when the piezoelectric crystals are excited.

### BACKGROUND OF THE INVENTION

The present invention relates to cataract surgery and more particularly to a phacoemulsification hand piece with an offset that produces an oscillatory motion.

The human eye functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light that can be transmitted to the retina. This deficiency is medically known as a cataract. An accepted treatment for cataracts is to surgically remove the cataract and replace the lens with an artificial intraocular lens (IOL). In the United States, the majority of cataractous lenses are removed using a surgical technique called phacoemulsification. During this procedure, a thin needle with a distal cutting tip is inserted into the diseased lens and vibrated ultrasonically. The vibrating cutting tip liquefies or emulsifies the lens so that the lens may be aspirated from the eye. The diseased lens, once removed, is replaced by an artificial intraocular lens (IOL).

A typical ultrasonic surgical device suitable for an ophthalmic procedure includes an ultrasonically driven hand piece, an attached cutting tip, an irrigating sleeve and an electronic control console. The hand piece assembly is attached to the control console by an electric cable or connector and flexible tubing. A surgeon controls the amount of ultrasound power that is delivered to the cutting tip of the hand piece and applied to tissue at any given time by depressing a foot pedal. Flexible tubing supplies irrigation fluid to and draws aspiration fluid from the eye through the hand piece assembly.

The operative part of the hand piece is a centrally located, hollow resonating bar or horn that is attached to a set of piezoelectric crystals. The crystals are controlled by the console and supply ultrasonic vibrations that drive both the horn and the attached cutting tip during phacoemulsification. The crystal/horn assembly is suspended within the hollow body or shell of the hand piece by flexible mountings. The hand piece body terminates in a reduced diameter portion or nose cone at the body's distal end. The nose cone is externally threaded to accept the irrigation sleeve. Likewise, the horn bore is internally threaded at its distal end to receive the external threads of the cutting tip. The irrigation sleeve also has an internally threaded bore that is screwed onto the external threads of the nose cone. The cutting tip is adjusted so that the tip projects only a predetermined amount past the open end of the irrigating sleeve.

In use, the ends of the cutting tip and the irrigating sleeve are inserted into a small incision in the cornea or sclera. One known cutting tip is ultrasonically vibrated along its longitudinal axis within the irrigating sleeve by the crystal-driven ultrasonic horn, thereby emulsifying the selected tissue. The hollow bore of the cutting tip communicates with the bore in the horn that in turn communicates with the aspiration line from the hand piece to the console. Other suitable cutting tips include piezoelectric elements that produce both longitudinal and torsional oscillations. One example of such a cutting tip is described in U.S. Pat. No. 6,402,769 (Boukhny).

A reduced pressure or vacuum source in the console draws or aspirates the emulsified tissue from the eye through the open end of the cutting tip, the cutting tip and horn bores and the aspiration line, and into a collection device. The aspiration of emulsified tissue is aided by a saline solution or other fluid that is injected into the surgical site through the small annular gap between the inside surface of the irrigating sleeve and the cutting tip.

One known surgical technique is to make the incision into the anterior chamber of the eye as small as possible in order to reduce the risk of induced post operative corneal curvature changes (astigmatism). These small incisions result in very tight wounds that squeeze the irrigating sleeve tightly against the vibrating tip. Friction between the irrigating sleeve and the vibrating tip generates heat. The risk of the tip overheating and burning tissue is reduced by the cooling effect of the aspirated fluid flowing inside the tip.

When the tip becomes occluded or clogged with emulsified tissue, the aspiration flow can be reduced or eliminated, which allows the tip to heat up. This practice also reduces cooling and results in a temperature increase, which may burn the tissue at the incision if left unchecked. In addition, during occlusion, a larger vacuum can build up in the aspiration tubing so that when the occlusion eventually breaks, a larger amount of fluid can be quickly suctioned from the eye, possibly resulting in the globe collapsing or other damage to the eye. Thus, it is important to dissipate the heat buildup at the incision to avoid tissue damage, and to prevent undesirable fluid surges from the eye during occlusion breaks.

Various heat generation reduction techniques are known. One way to reduce the amount of generated heat is to lessen the friction coefficient of the material that the vibrating phacoemulsification needle contacts. For instance, instead of allowing the needle to touch the rather sticky infusion sleeve made of liquid injection molded silicone, an intervening tubing made from a lower friction material such as polyimide may be employed to significantly reduce the amount of heat generated by friction. Another way is to divert irrigation flow though a bypass opening in the phacoemulsification needle in the event that the tip port of the needle becomes occluded by lens fragments. That way, irrigation flow continues to cool the needle despite the occlusion. Reduction in heat generation may also be realized by lowering the vibration amplitude and/or reducing the operating duty cycle of the phacoemulsification tip.

Another way to reduce heat build up is with the use of torsional movement of the hand piece tip. Torsional movement involves a twisting and preferably rotating movement of the tip about the longitudinal axis of the tip. Such torsional movement may be accomplished by the ultrasonic hand piece having a programmable ultrasound driver capable of producing both a torsional frequency drive signal and a longitudinal frequency drive signal. Such hand pieces are well-known to those in the art, with one example being described in U.S. Pat. No. 6,028,387. Torsional movement also allows for more effective lens removal

Instead of traditional torsional movement, an oscillatory movement of the hand piece tip can produce similar beneficial results. In addition to lessening heat build up, an oscillatory movement results in more effective lens removal. Like a torsional movement, an oscillatory movement produces less repulsion of lens fragments at the cutting tip. The reduction in repulsion allows for more effective aspiration of lens fragments. It would be desirable to have a hand piece that produces an oscillatory movement of the cutting tip.

### SUMMARY OF THE INVENTION

In one embodiment consistent with the principles of the present invention, the present invention is an ultrasonic hand piece including a horn, piezoelectric crystals, and a cutting tip. The piezoelectric crystals and cutting tip are coupled to the horn. A centerline of the piezoelectric crystals is offset from a center line of the cutting tip such that oscillatory movement is produced in the cutting tip when the piezoelectric crystals are excited.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 depicts a traditional ultrasonic hand piece.

FIG. 2 depicts an offset ultrasonic hand piece according to the principles of the present invention.

FIG. 3 depicts an offset ultrasonic hand piece according to the principles of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

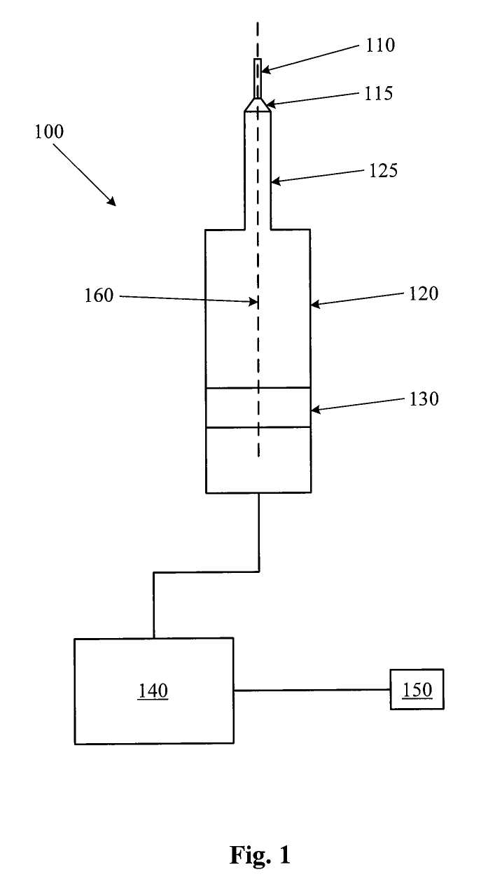

FIG. 1 depicts a traditional ultrasonic hand piece. In FIG. 1, hand piece 100 is coupled to console 140. Console 140 is coupled to foot switch 150. Hand piece 100 has a cutting tip 110, a horn 120, and a set of piezoelectric crystals 130. A tip interface 115 connects cutting tip 110 to a reduced diameter portion 125 of horn 120. A centerline 160 of the hand piece 100 is also depicted.

Tip 110 is typically a thin needle made of titanium or stainless steel that is designed to emulsify a lens when vibrated ultrasonically. Tip 110 is typically cylindrical in shape, has a small diameter of about 20-30 gauge, and has a length suitable for removal of a lens when inserted into the anterior chamber of the eye. Tip 110 has a central longitudinal axis that passes through its center of gravity in a longitudinal direction. For example, when tip 110 approximates a cylinder, its central longitudinal axis is the central axis of the cylinder. The central longitudinal axis of tip 110 is depicted by dashed line (center line) 160. In hand piece 100, centerline 160 passes though the center of tip 110 and the center of piezoelectric crystals 130. In this manner, tip 110 is aligned with piezoelectric crystals 130. Such an alignment typically causes a longitudinal movement in tip 110 when piezoelectric crystals 130 are excited.

Horn 120 is typically made of a rigid material suitable for medical use (such as a titanium alloy). Horn 120 has a reduced diameter section 125 that is connected to a tip interface 115. Tip interface 115 typically has a threaded connection that accepts tip 110. In this manner tip 110 is screwed onto horn 120 at tip interface 115. This provides a rigid connection between tip 110 and horn 120 so that vibration can be transmitted from horn 120 to tip 110.

Piezoelectric crystals 130 supply ultrasonic vibrations that drive both the horn 120 and the attached cutting tip 110 during phacoemulsification. Piezoelectric crystals 130 are affixed to horn 120. Crystals 130 are typically ring shaped, resembling a hollow cylinder and constructed from a plurality of crystal segments. When excited by a signal from console 140, crystals 130 resonate, producing vibration in horn 120. Typically, this vibration produces longitudinal movement in tip 110.

Console 140 includes a signal generator that produces a signal to drive piezoelectric crystals 130. Console 140 has a suitable microprocessor, micro-controller, computer, or digital logic controller to control the signal generator. In operation, console 140 produces a signal that drives piezoelectric crystals 130. Piezoelectric crystals 130, when excited, cause horn 120 to vibrate. Tip 110, connected to horn 120, also vibrates. When tip 110 is inserted into the anterior chamber of the eye and vibrated, it acts to emulsify a cataractous lens.

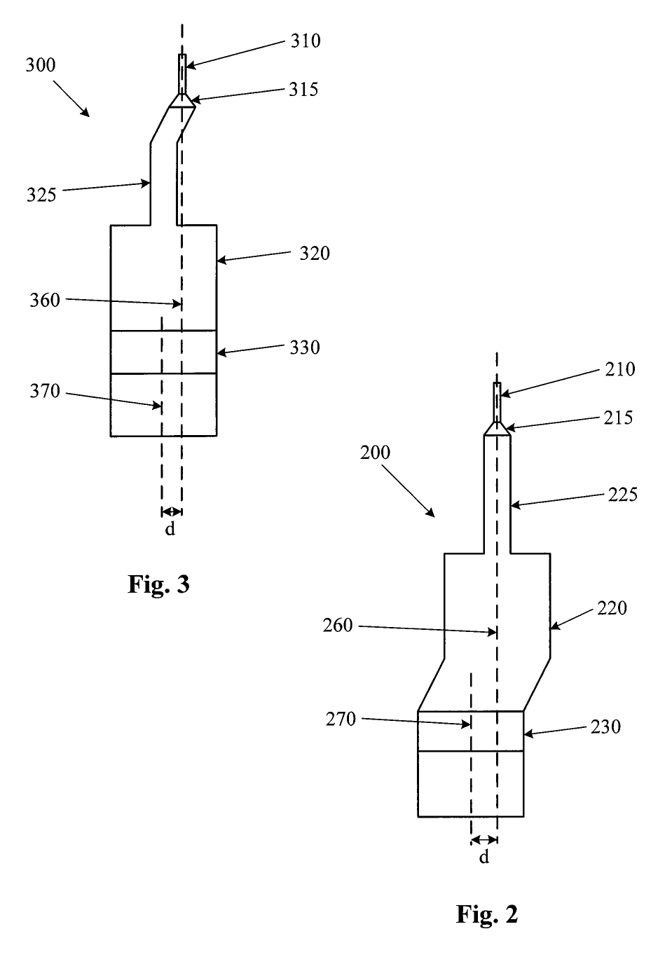

FIG. 2 depicts an offset ultrasonic hand piece according to the principles of the present invention. The components of FIG. 2 perform in a similar manner and have similar characteristics as like components in FIG. 1. In FIG. 2, tip 210 is offset from piezoelectric crystals 230. This offset is accomplished by a bend or step in horn 220. As such, tip center line 260 is offset from crystal center line 270 by a distance “d.” This distance can be small to produce a small oscillatory movement of tip 210, or it can be large to produce a larger oscillatory movement of tip 210. As such, the distance “d” is proportional to the amount of oscillatory movement seen at tip 210. Such a configuration causes tip 210 to oscillate or wobble in a side to side or circular fashion. Vibration from piezoelectric crystals 230 is transferred to horn 220 such that tip 210 wobbles or oscillates.

Tip center line 260 of tip 210 generally passes through the center of gravity of tip 210. Likewise, crystal center line 270 generally passes through the center of piezoelectric crystals 230. When piezoelectric crystals 230 are arranged in a ring, crystal center line 270 passes through the center of that ring. Tip center line 260 is generally parallel to crystal center line 270. However, these two lines need not be parallel. For example, tip center line 260 may be at a small angle with respect to crystal center line 270. In this manner, tip 210 may extend from tip interface 215 at a slight angle in addition to being offset from piezoelectric crystals 230.

FIG. 3 depicts an offset ultrasonic hand piece according to the principles of the present invention. The components of FIG. 3 perform in a similar manner and have similar characteristics as like components in FIG. 1. In FIG. 3, tip 310 is offset from piezoelectric crystals 330. This offset is accomplished by a bend or step in the reduced diameter section 325 of horn 320. As such, tip center line 360 is offset from crystal center line 370 by a distance “d.” This distance can be small to produce a small oscillatory movement of tip 310, or it can be large to produce a larger oscillatory movement of tip 310. As such, the distance “d” is proportional to the amount of oscillatory movement seen at tip 310. Such a configuration causes tip 310 to oscillate or wobble in a side to side or circular fashion. Vibration from piezoelectric crystals 330 is transferred to horn 320 such that tip 310 wobbles or oscillates.

Tip center line 360 of tip 310 generally passes through the center of gravity of tip 310. Likewise, crystal center line 370 generally passes through the center of piezoelectric crystals 330. When piezoelectric crystals 330 are arranged in a ring, crystal center line 370 passes through the center of that ring. Tip center line 360 is generally parallel to crystal center line 370. However, these two lines need not be parallel. For example, tip center line 360 may be at a small angle with respect to crystal center line 370. In this manner, tip 30 may extend from tip interface 315 at a slight angle in addition to being offset from piezoelectric crystals 370.

From the above, it may be appreciated that the present invention provides an offset ultrasonic hand piece useful for the removal of a cataractous lens. In the present invention, the cutting tip is offset from the piezoelectric crystals that drive the hand piece. Such an offset produces oscillatory movement of the hand piece tip. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ultrasonic hand piece comprising:
a horn;
piezoelectric crystals coupled to the horn; and
a cutting tip coupled to the horn;
wherein a centerline of the piezoelectric crystals is offset from a center line of the cutting tip such that oscillatory movement is produced in the cutting tip when the piezoelectric crystals are excited.

2. The hand piece of claim 1 wherein the horn has a reduced diameter segment.

3. The hand piece of claim 2 wherein the reduced diameter segment is bent to produce the offset.

4. The hand piece of claim 1 wherein the horn is bent to produce the offset.
